(19) United States
(12) Patent Application Publication
(10) Pub. No.: US 2025/0257163 A1
(43) Pub. Date: Aug. 14, 2025

(54) SPIROPYRAN-BASED PHOTO-HYDROGEL

(71) Applicants: Toyota Motor Engineering & Manufacturing North America, Inc., Plano, TX (US); The Board of Trustees of the University of Illinois, Urbana, IL (US)

(72) Inventors: Jiayu Liu, Urbana, IL (US); Lihong Lao, Urbana, IL (US); Wonsik Eom, Champaign, IL (US); Paul Vannest Braun, Champaign, IL (US); Shailesh N. Joshi, Ann Arbor, MI (US); Ercan Mehmet Dede, Ann Arbor, MI (US); Jae Seung Lee, Ann Arbor, MI (US); Frederico Marcolino Quintao Severgnini, Ann Arbor, MI (US)

(73) Assignees: Toyota Motor Engineering & Manufacturing North America, Inc., Plano, TX (US); Toyota Jidosha Kabushiki Kaisha, Toyota-shi Aichi-ken (JP); The Board of Trustees of the University of Illinois, Urbana, IL (US)

(21) Appl. No.: 18/436,206

(22) Filed: Feb. 8, 2024

(51) Int. Cl. C08F 220/56; A61L 27/18; A61L 27/52; C08J 3/075

(52) U.S. Cl. CPC C08F 220/56; A61L 27/18; A61L 27/52; C08J 3/075; A61L 2430/30; C08J 2333/26

(57) ABSTRACT

Functionalized spiropyran-based photo-hydrogels that include at least two different spiropyrans are disclosed herein. The disclosed photo-hydrogels may include two spiropyrans with differing chemical properties. In some variations, the spiropyran-based photo-hydrogel includes two functionalized spiropyrans, including a first functionalized spiropyran having a net positive charge and a second functionalized spiropyran having a net negative charge. The net positive and net negative charges are equal and opposite, such that the spiropyran-based photo-hydrogel has an overall net zero charge. In the absence of light, the spiropyrans are in their corresponding merocyanine forms and the photo-hydrogel has a high degree of aggregation. Irradiation, such as exposure to visible light, causes photo-isomerization of the merocyanines into spiropyrans, resulting in an increase in the amount of swelling of the photo-hydrogel. In some implementations, the disclosed photo-hydrogels may be used in a variety of applications such as light-controlled micropumps, artificial eye muscles, cameras, and drug delivery systems.

### TECHNICAL FIELD

The present disclosure generally relates to photo-hydrogels, more particularly spiropyran-based photo-hydrogels.

### BACKGROUND

Spiropyrans are photochromatic compounds used in a variety of applications. Among the advantages of using spiropyrans in applications requiring photochromaticy is their extremely versatile photoconversion properties. This results from the sensitivity of spiropyrans to a large variety of stimuli, including heat, solvent polarity, protonation, metal complexation, and redox-induced electrodimerization processes. The photoisomers of spiropyrans are known as merocyanines.

Photo-hydrogels that include spiropyrans are known to expand or contract in response to light. However, the amount of volume change during expansion or contraction is generally small, which limits the practical applications of existing spiropyran-based photo-hydrogels. Known spiropyran-based photo-hydrogels include a single type of spiropyran.

The present disclosure addresses the limitations of known spiropyran-based photo-hydrogels.

### SUMMARY

Functionalized spiropyran-based photo-hydrogels that include at least two different spiropyrans are disclosed herein. The disclosed photo-hydrogels may include two spiropyrans with differing chemical properties. This may result in an increase in the amount of swelling of the photo-hydrogel upon irradiation, such as exposure to visible light. In some implementations, the disclosed photo-hydrogels may be used in a variety of applications such as light-controlled micropumps, artificial eye muscles, cameras, and drug delivery systems.

In some variations, the photo-hydrogel has the following structure (I), shown in its merocyanine form (formula):

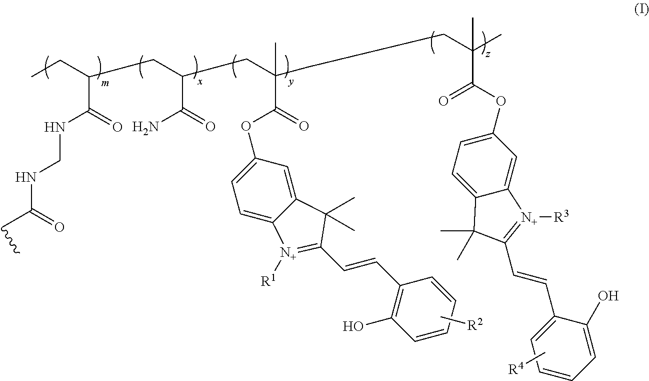

In some variations, the spiropyran-based photo-hydrogel includes two functionalized spiropyrans, including a first functionalized spiropyran having a net positive charge and a second functionalized spiropyran having a net negative charge. The net positive and net negative charges are equal and opposite, such that the spiropyran-based photo-hydrogel has an overall net zero charge. The equal and opposite charges of the functionalized spiropyrans creates electrostatic attraction between the distinct functionalized spiropyran units. Prior to irradiation, the functionalized spiropyrans are in their merocyanine forms. The electrostatic interactions between charged functionalized merocyanines causes the merocyanines to structurally align. When structurally aligned, the conjugation within the merocyanines causes further structural aggregation that leads to increased density and thus a reduced volume of the photo-hydrogel.

Upon irradiation, functionalized merocyanines undergo ring-closing to form corresponding spiropyrans. Irradiation causes the aggregated merocyanines to photoisomerize and de-aggregate. The result of this de-aggregation is swelling of the photo-hydrogel.

In some variations, the functional group R¹ in (I) is selected from the group consisting of alkylsulfonates and arylsulfonates. In some variations, the functional group R¹ in (I) is selected from the group consisting of C₁-C₈ unbranched alkylsulfonates, C₁-C₈ branched alkylsulfonates, substituted monoarylsulfonates, and unsubstituted monoarylsulfonates.

In some alternate variations, the functional group R¹ in (I) is selected from the group consisting of alkylphosphates and arylphosphates. In some variations, the functional group R¹ in (I) is selected from the group consisting of C₁-C₈ unbranched alkylphosphates, C₁-C₈ branched alkylphosphates, substituted monoarylphosphates, and unsubstituted monoarylphosphates.

In some variations, the functional group R² in (I) is selected from the group consisting of alkylsulfonates and arylsulfonates. In some variations, the functional group R² in (I) is selected from the group consisting of C₁-C₈ unbranched alkylsulfonates, C₁-C₈ branched alkylsulfonates, substituted monoarylsulfonates, and unsubstituted monoarylsulfonates.

In some alternate variations, the functional group R² in (I) is selected from the group consisting of alkylphosphates and arylphosphates. In some variations, the functional group R² in (I) is selected from the group consisting of C₁-C₈ unbranched alkylphosphates, C₁-C₈ branched alkylphosphates, substituted monoarylphosphates, and unsubstituted monoarylphosphates.

In some other alternate variations, the functional group R² in (I) is selected from the group p consisting of substituted polyarylsulfonates, unsubstituted polyarylsulfonates, substituted polyarylphosphates, and unsubstituted polyarylphosphates.

In some variations, the functional group R³ in (I) is selected from the group consisting of alkylammoniums and arylammoniums. In some variations, the functional group R³ in (I) is selected from the group consisting of C₁-C₈ unbranched alkylammoniums, C₁-C₈ branched alkylammoniums, substituted monoarylammoniums, and unsubstituted monoarylammoniums.

In some variations, the functional group R³ in (I) is selected from the group consisting of alkylammoniums and arylammoniums. In some variations, the functional group R³ in (I) is selected from the group consisting of C₁-C₈ unbranched alkylammoniums, C₁-C₈ branched alkylammoniums, substituted monoarylammoniums, and unsubstituted monoarylammoniums.

In at least one variation, the functional group R³ in (I) is selected from the group consisting of alkylimidazoliums and arylimidazoliums. In some variations, the functional group R³ in (I) is selected from the group consisting of C₁-C₈ unbranched alkylimidazoliums, C₁-C₈ branched alkylimidazoliums, substituted monoarylimidazoliums, and unsubstituted monoarylimidazoliums.

In still other variations, the functional group R³ in (I) is selected from the group consisting of alkylpyridiniums and arylpyridiniums. In some variations, the functional group R³ in (I) is selected from the group consisting of C₁-C₈ unbranched alkylpyridiniums, C₁-C₈ branched alkylpyridiniums, substituted monoarylpyridiniums, and unsubstituted mono arylpyridiniums.

In some variations, the functional group R⁴ in (I) is selected from the group consisting of alkylsulfonates and arylsulfonates. In some variations, the functional group R⁴ in (I) is selected from the group consisting of C₁-C₈ unbranched alkylsulfonates, C₁-C₈ branched alkylsulfonates, substituted monoarylsulfonates, and unsubstituted monoarylsulfonates.

In some alternate variations, the functional group R⁴ in (I) is selected from the group consisting of alkylphosphates and arylphosphates. In some variations, the functional group R⁴ in (I) is selected from the group consisting of C₁-C₈ unbranched alkylphosphates, C₁-C₈ branched alkylphosphates, substituted monoarylphosphates, and unsubstituted monoarylphosphates.

In some other alternate variations, the functional group R⁴ in (I) is selected from the group consisting of substituted polyarylsulfonates, unsubstituted polyarylsulfonates, substituted polyarylphosphates, and unsubstituted polyarylphosphates.

In some variations, the value of ‘x’ in (I) is the reference and is set or equal to 100, the value of ‘m’ in (I) is less than 5, e.g., 1 or 2, and the values of ‘x’ and ‘y’ are equal to each other and less than 16, e.g., between 4 and 8, inclusive (i.e., x and y can equal 4 or 8).

It should be understood that ‘m’ is a hydrogel crosslinker and ‘x’ is a hydrogel monomer. In addition, ‘m’ can be poly(ethylene glycol) dimethacrylate and ‘x’ can be a non-charged hydrogel such as N-isopropylacrylamide, hydroxyalkyl methacrylates, and N-hydroxyalkyl acrylamides, among others.

In some variations, the photo-hydrogel has the following structure (II):

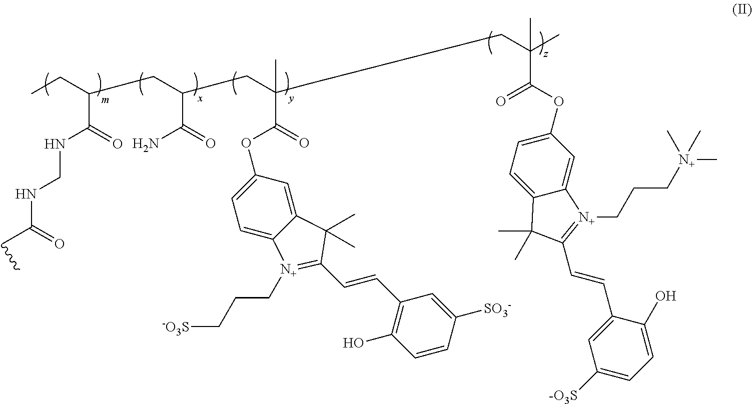

with m, x, y, and z as described above for the structure (I).

These and other features of the disclosed spiropyran-based photo-hydrogels will become apparent from the following detailed description when read in conjunction with the figures and examples, which are exemplary, not limiting.

### BRIEF DESCRIPTION OF THE DRAWINGS

The present teachings will become more fully understood from the detailed description and the accompanying drawings, wherein:

FIG. 1 illustrates the mechanism of deaggregation of the spiropyran units of a photo-hydrogel;

FIG. 2 provides a plot of the percent of area change as a function of pH using white light for two different concentrations of spiropyrans;

FIG. 3 illustrates how swelling is observed in an exemplary photo-hydrogel;

FIGS. 4A-4C illustrates an exemplary photo-hydrogel micropump, where FIG. 4A shows a schematic illustration of the photo-hydrogel micropump, FIG. 4B illustrates an unactuated photo-hydrogel micropump, and FIG. 4C illustrates an actuated photo-hydrogel micropump; and

FIG. 5 illustrates an exemplary photo-hydrogel artificial eye muscle.

It should be noted that the figures set forth herein are intended to exemplify the general characteristics of the disclosed spiropyran-based photo-hydrogels, for the purpose of the description of certain aspects. The figures may not precisely reflect the characteristics of any given aspect and are not necessarily intended to define or limit specific forms or variations within the scope of this technology.

### DETAILED DESCRIPTION

Functionalized spiropyran-based photo-hydrogels that include at least two different spiropyrans are disclosed herein. The disclosed photo-hydrogels may include two spiropyrans with differing chemical properties. This may result in an increase in the amount of swelling of the photo-hydrogel upon irradiation, such as exposure to visible light. In some implementations, the disclosed photo-hydrogels may be used in a variety of applications such as light-controlled micropumps, artificial eye muscles, cameras, and drug delivery systems.

In some variations, the photo-hydrogel includes a polymer with the following structure (I), shown in its merocyanine form (formula):

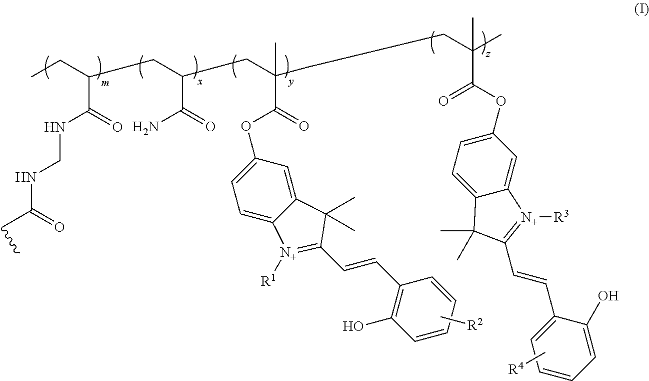

In some variations, the spiropyran-based photo-hydrogel includes two functionalized spiropyrans, including a first functionalized spiropyran having a net positive charge and a second functionalized spiropyran having a net negative charge. The net positive and net negative charges are equal and opposite, such that the spiropyran-based photo-hydrogel has an overall net zero charge. The equal and opposite charges of the functionalized spiropyrans creates electrostatic attraction between the distinct functionalized spiropyran units. Prior to irradiation, the functionalized spiropyrans are in their merocyanine forms. The electrostatic interactions between charged functionalized merocyanines causes the merocyanines to structurally align. When structurally aligned, the conjugation within the merocyanines causes further structural aggregation that leads to increased density and thus a reduced volume of the photo-hydrogel.

Upon irradiation, functionalized merocyanines undergo ring-closing to form corresponding spiropyrans. Irradiation causes the aggregated merocyanines to photoisomerize and de-aggregate. The result of this de-aggregation is swelling of the photo-hydrogel.

In some variations, the functional group R¹ in (I) is selected from the group consisting of alkylsulfonates and arylsulfonates. In some variations, the functional group R¹ in (I) is selected from the group consisting of C₁-C₈ unbranched alkylsulfonates, C₁-C₈ branched alkylsulfonates, substituted monoarylsulfonates, and unsubstituted monoarylsulfonates.

In some alternate variations, the functional group R¹ in (I) is selected from the group consisting of alkylphosphates and arylphosphates. In some variations, the functional group R¹ in (I) is selected from the group consisting of C₁-C₈ unbranched alkylphosphates, C₁-C₈ branched alkylphosphates, substituted monoarylphosphates, and unsubstituted monoarylphosphates.

In some variations, the functional group R² in (I) is selected from the group consisting of alkylsulfonates and arylsulfonates. In some variations, the functional group R² in (I) is selected from the group consisting of C₁-C₈ unbranched alkylsulfonates, C₁-C₈ branched alkylsulfonates, substituted monoarylsulfonates, and unsubstituted monoarylsulfonates.

In some alternate variations, the functional group R² in (I) is selected from the group consisting of alkylphosphates and arylphosphates. In some variations, the functional group R² in (I) is selected from the group consisting of C₁-C₈ unbranched alkylphosphates, C₁-C₈ branched alkylphosphates, substituted monoarylphosphates, and unsubstituted monoarylphosphates.

In some other alternate variations, the functional group R² in (I) is selected from the group consisting of substituted polyarylsulfonates, unsubstituted polyarylsulfonates, substituted polyarylphosphates, and unsubstituted polyarylphosphates.

In some variations, the functional group R³ in (I) is selected from the group consisting of alkylammoniums and arylammoniums. In some variations, the functional group R³ in (I) is selected from the group consisting of C₁-C₈ unbranched alkylammoniums, C₁-C₈ branched alkylammoniums, substituted monoarylammoniums, and unsubstituted monoarylammoniums.

In at least one variation, the functional group R³ in (I) is selected from the group consisting of alkylimidazoliums and arylimidazoliums. In some variations, the functional group R³ in (I) is selected from the group consisting of C₁-C₈ unbranched alkylimidazoliums, C₁-C₈ branched alkylimidazoliums, substituted monoarylimidazoliums, and unsubstituted monoarylimidazoliums.

In still other variations, the functional group R³ in (I) is selected from the group consisting of alkylpyridiniums and arylpyridiniums. In some variations, the functional group R³ in (I) is selected from the group consisting of C₁-C₈ unbranched alkylpyridiniums, C₁-C₈ branched alkylpyridiniums, substituted monoarylpyridiniums, and unsubstituted mono arylpyridiniums.

In some variations, the functional group R⁴ in (I) is selected from the group consisting of alkylsulfonates and arylsulfonates. In some variations, the functional group R⁴ in (I) is selected from the group consisting of C₁-C₈ unbranched alkylsulfonates, C₁-C₈ branched alkylsulfonates, substituted monoarylsulfonates, and unsubstituted monoarylsulfonates.

In some alternate variations, the functional group R⁴ in (I) is selected from the group consisting of alkylphosphates and arylphosphates. In some variations, the functional group R⁴ in (I) is selected from the group consisting of C₁-C₈ unbranched alkylphosphates, C₁-C₈ branched alkylphosphates, substituted monoarylphosphates, and unsubstituted monoarylphosphates.

In some other alternate variations, the functional group R⁴ in (I) is selected from the group consisting of substituted polyarylsulfonates, unsubstituted polyarylsulfonates, substituted polyarylphosphates, and unsubstituted polyarylphosphates.

In some variations, the value of ‘x’ in (I) is the reference and is set or equal to 100, the value of ‘m’ in (I) is less than 5, e.g., 1 or 2, and the values of ‘x’ and ‘y’ are equal to each other and less than 16, e.g., between 4 and 8, inclusive.

It should be understood that ‘m’ is a hydrogel crosslinker and ‘x’ is a hydrogel monomer. In addition, ‘m’ can be poly(ethylene glycol) dimethacrylate and ‘x’ can be a non-charged hydrogel such as N-isopropylacrylamide, hydroxyalkyl methacrylates, and N-hydroxyalkyl acrylamides, among others.

A general method for synthesizing photo-hydrogels according to the teachings of the present disclosure includes dissolving water soluble spiropyrans, hydrogel monomers, and a crosslinker in a mixture of an organosulfur compound, sparging the solution, and then catalyzing the sparged solution before transferring to a mold where polymerization occurs. For example, in some variations the photo-hydrogel according to the teachings of the present disclosure were synthesized by dissolving 18% w/v of water soluble spiropyrans, hydrogel monomers and crosslinker were in 4:1 v/v dimethyl sulfoxide (DMSO): Milli-Q purified water to form a liquid solution. The solution was then sparged with N₂ for 15 mins to remove O₂, and the catalyst tetramethylethylenediamine (TEMED) and initiator ammonium persulfate solution were added to the solution before vortexing the solution and transferring to a glass mold with a 0.4 mm thick plastic tape spacer. Polymerization was carried out at room temperature for 2.5 hours in the mold and the obtained hydrogel was incubated in pH 2.0 water overnight before further study.

In one particular example, acrylamide (AAm, 100 mg, 100 mol %), SP1 (42.3 mg, 4 mol %), SP2 (35.4 mg, 4 mol %), and N,N-methylenebisacrylamide (BIS, 4.7 mg, 2.16 mol % (2% crosslink of all monomers)) were completely dissolved in DMSO (812 μL): MilliQ water (121 μL). The solution was then sparged with N₂ for 15 mins to remove O₂, and TEMED (13.3 μL) and freshly made initiator of 10 wt % ammonium persulfate solution (APS, 82 μL, 8.2 mg, 4.5 wt % of all monomers) were then quickly added to the solution and vortexed before transferring to the mold. The polymerization was carried out at room temperature for 2.5 hours in the mold and the obtained hydrogel was soaked in isopropanol and then pH 2.0 water containing 10 mM of HCl. After washing with pH 2.0 water 4 times every 30 mins to remove all solvents and unreacted monomers, the hydrogel was incubated in pH 2.0 water overnight at room temperature in dark to allow isomerization and aggregation of merocyanines. The as prepared hydrogel was directly used without further characterization.

While not being bound by theory, it should be understood that the concentrations (i.e., ‘y’ and ‘z’) of SP1 and SP2 in the hydrogel will affect the swelling ratio, and the higher the concentration(s), the larger expansion the hydrogel will have. However, with larger expansion, the hydrogel generally exhibits or has an increase in fragility due to higher water content and weaker chain interactions. Therefore, 4% of each spiropyran was chosen in order to the hydrogel to maintain adequate strength and exhibit a high swelling ratio.

In addition, the concentration of monomers in the solution prepared before polymerization will affect swelling ratio, i.e., the lower the concentration, the less entanglement of hydrogel networks and thus an increase in expansion. However, when the concentration is below around 18% w/v, a uniform hydrogel network maty not form and thus 18% w/v of the monomers was chosen for polymerization.

In addition, the contraction process will be accelerated under higher temperature, e.g., the contraction process to 1.5 hours at 40° C. and 5.5 hours at room temperature.

In some variations, the photo-hydrogel includes a polymer with the following structure or formula (II):

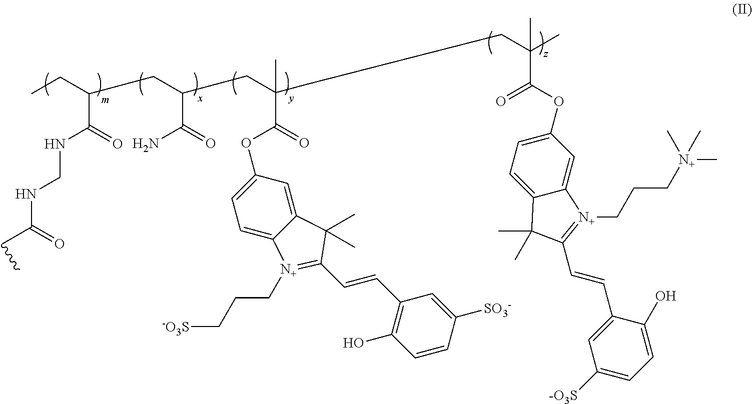

with m, x, y, and z as described above for the structure (I).

When the photo-hydrogel is not exposed to light, the spiropyrans are closely stacked together, thereby resulting in a relatively small hydrogel volume. However, when light is incident on the photo-hydrogel, not only do the spiropyrans individually expand, but the two different spiropyrans deaggregate and are no longer closely stacked together. This results in greater expansion of the photo-hydrogel than would be observed in a photo-hydrogel having only a single type of spiropyran.

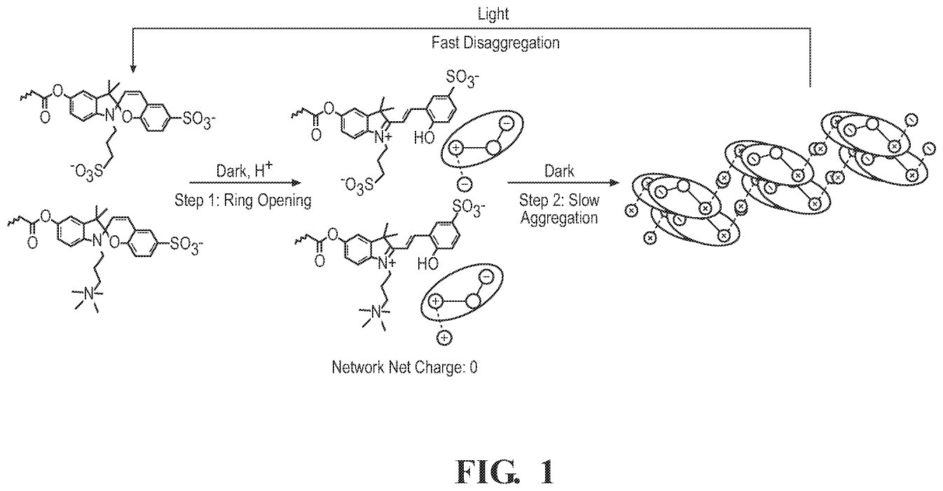

FIG. 1 illustrates the mechanism of deaggregation of the spiropyran units of a photo-hydrogel. Deaggregation of the spiropyran units occurs quickly upon exposure of the spiropyrans to light. Alternately, deaggregation may be carried out more slowly under protic acidic conditions in the absence of light.

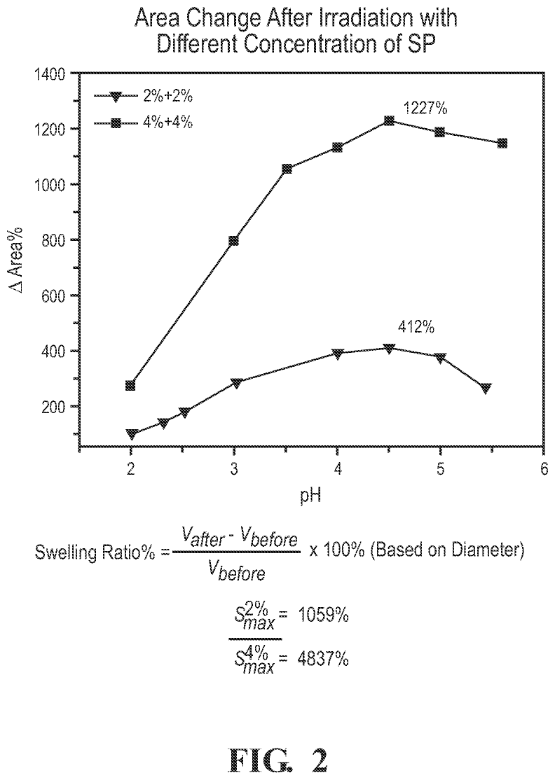

FIG. 2 provides a plot of the percent of area change as a function of pH using white light for two different concentrations of spiropyrans. As shown in FIG. 2, where the concentration of each spiropyran is 2% by number (i.e., for every 100 ‘x’ monomers, there are 2 spiropyrans), the maximum observed swelling is over 1000% of the original volume. Also as shown in FIG. 2, where the concentration of each spiropyran is 4% by number, the maximum observed swelling is over 4000% of the original volume.

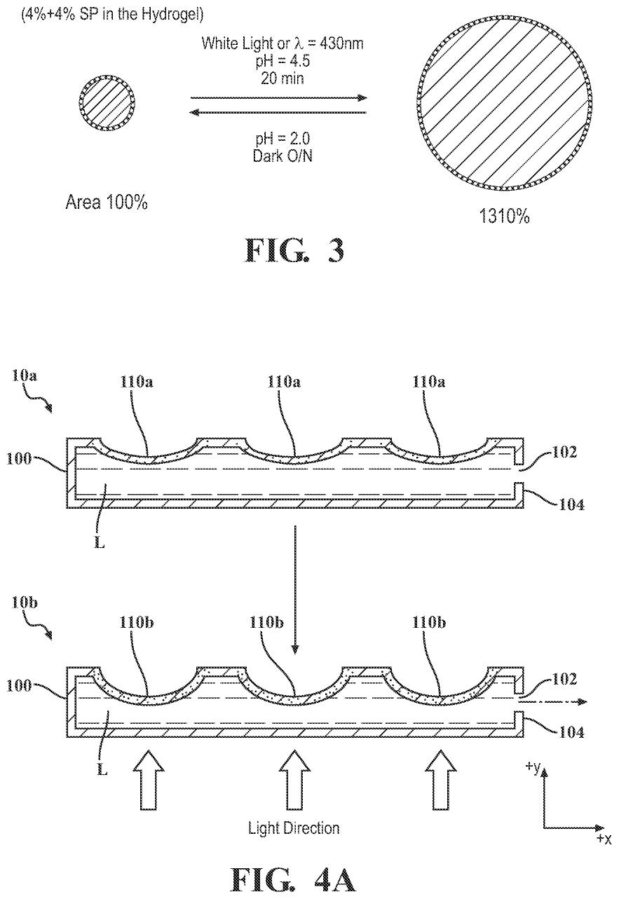

FIG. 3 illustrates how swelling is observed in an exemplary photo-hydrogel upon exposure to white or blue light at pH 4.5 for 20 min. As shown in FIG. 3, the swelling may be reversed by placing the photo-hydrogel in the dark.

The disclosed photo-hydrogel may be used in a variety of applications, including light-controlled micropumps, artificial eye muscles, cameras, and drug delivery systems.

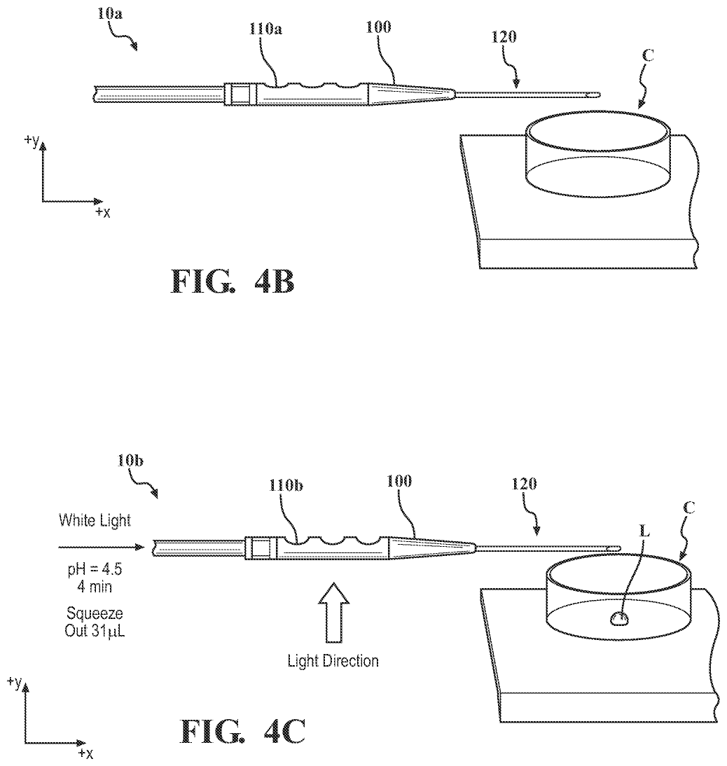

FIGS. 4A-4C illustrate a light-controlled micropump 10 that uses the disclosed photo-hydrogel. With reference to FIG. 4A, the micropump 10 in a first state 10a is shown above (+y direction) the micropump 10 in a second state 10b. The micropump 10 includes a rigid enclosure 100 with an opening 102 at a side wall 104 and photo-hydrogel layers 110 disposed or included as part of an upper (+y direction) wall. The bottom (−y direction) of the rigid enclosure 100 is transparent to the wavelength of light generated by a light source used to activate the photo-hydrogel layers 110. Light from the light source passes through the rigid enclosure 100 and the water, and the photo-hydrogel layers 110 in a first state 110a are thereby exposed to the light. The photo-hydrogel layers 110 expand in the direction towards the light source (−y direction) into a second state 110b such that a liquid ‘L’ in the micropump 10 is forced through the opening 102 in the side wall 104 as illustrated by the micropump in the second state 10b. FIG. 4B shows the micropump 10 in the form of a syringe with the photo-hydrogel layers 110 in the first state and FIG. 4C shows the syringe in FIG. 4B with the photo-hydrogel layers 110 exposed to light and in the second state 110b such that liquid Lis pumped from the syringe and into a container ‘C’.

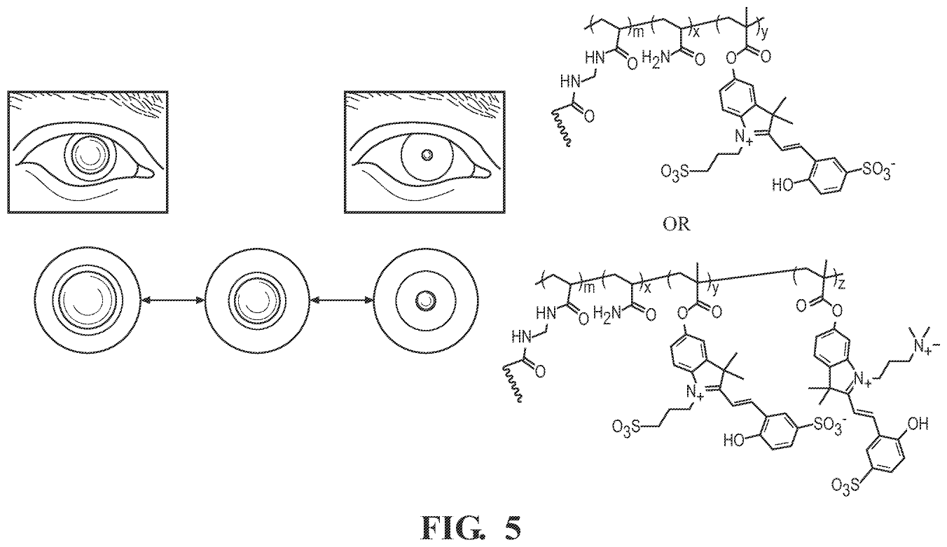

FIG. 5 illustrates an exemplary artificial eye muscle composed of a spiropyran-based photo-hydrogel. The artificial eye muscle is shaped like and functions like a biological eye muscle. The photo-hydrogel is attached to a rigid outer ring. When the photo-hydrogel is exposed to light, it expands inward. This reduces the diameter of the pupil. No external actuator, such as a heater, is required. In some variations, the artificial eye muscle may be worn like a contact lens on the corneal surface. An inner barrier layer may be used to isolate the photo-hydrogel actuator from the eye. This inner barrier layer may also have filtering capabilities to filter out undesirable wavelengths of light.

In view of the above teachings it should be understood that a photo-hydrogel is provided that exhibits enhanced expansion and contraction compared to traditional hydrogels and that such photo-hydrogels have uses such as light-controlled micropumps, artificial eye muscles, camera lenses, tactile surfaces, and/or drug delivery systems.

The preceding description is merely illustrative in nature and is in no way intended to limit the disclosure, its application, or uses. As used herein, the phrase at least one of A, B, and C should be construed to mean a logical (A or B or C), construing “or” to have its non-exclusive logical meaning. It should be understood that the various steps within a method may be executed in different order without altering the principles of the present disclosure. Disclosure of ranges includes disclosure of all ranges and subdivided ranges within the entire range.

The headings (such as “Background” and “Summary”) and sub-headings used herein are intended only for general organization of topics within the present disclosure and are not intended to limit the disclosure of the technology or any aspect thereof. The recitation of multiple forms or variations having stated features is not intended to exclude other forms or variations having additional features, or other forms or variations incorporating different combinations of the stated features.

As used herein the terms “about” and “generally” when related to numerical values herein refers to known commercial and/or experimental measurement variations or tolerances for the referenced quantity. In some variations, such known commercial and/or experimental measurement tolerances are ±10% of the measured value, while in other variations such known commercial and/or experimental measurement tolerances are ±5% of the measured value, while in still other variations such known commercial and/or experimental measurement tolerances are ±2.5% of the measured value, and in still other variations, such known commercial and/or experimental measurement tolerances are ±1% of the measured value.

As used herein, the terms “comprise” and “include” and their variants are intended to be non-limiting, such that recitation of items in succession or a list is not to the exclusion of other like items that may also be useful in the devices and methods of this technology. Similarly, the terms “can” and “may” and their variants are intended to be non-limiting, such that recitation that a form or variation can or may comprise certain elements or features does not exclude other forms or variations of the present technology that do not contain those elements or features.

The broad teachings of the present disclosure can be implemented in a variety of forms. Therefore, while this disclosure includes particular examples, the true scope of the disclosure should not be so limited since other modifications will become apparent to the skilled practitioner upon a study of the specification and the following claims. Reference herein to one aspect, or various aspects means that a particular feature, structure, or characteristic described in connection with a form or variation is included in at least one form or variation. The appearances of the phrase “in one variation” or “in one form” (or variations thereof) are not necessarily referring to the same form or variation. It should be also understood that the various method steps discussed herein do not have to be carried out in the same order as depicted, and not each method step is required in each form or variation.

The foregoing description of the forms or variations has been provided for purposes of illustration and description. It is not intended to be exhaustive or to limit the disclosure. Individual elements or features of a particular form or variation are generally not limited to that particular form or variation, but, where applicable, are interchangeable and can be used in a selected form or variation, even if not specifically shown or described. The same may also be varied in many ways. Such variations should not be regarded as a departure from the disclosure, and all such modifications are intended to be included within the scope of the disclosure.

While particular forms or variations have been described, alternatives, modifications, variations, improvements, and substantial equivalents that are or may be presently unforeseen may arise to applicants or others skilled in the art. Accordingly, the appended claims as filed and as they may be amended, are intended to embrace all such alternatives, modifications, variations, improvements, and substantial equivalents.

What is claimed is:

1. A photo-hydrogel comprising a polymer of formula (I): 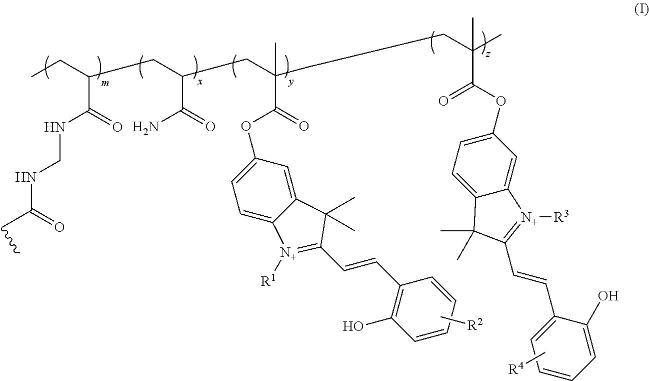
where R¹ is different from R³, a net charge of R¹ is negative and a net charge of R³ is positive, and when x=100, m<5 and x=y<16.

2. The photo-hydrogel according to claim 1, wherein R¹ is selected from the group consisting of alkylsulfonates and arylsulfonates.

3. The photo-hydrogel according to claim 2, wherein R¹ is selected from the group consisting of C₁-C₈ unbranched alkylsulfonates, C₁-C₈ branched alkylsulfonates, substituted monoarylsulfonates, and unsubstituted monoarylsulfonates.

4. The photo-hydrogel according to claim 1, wherein R¹ is selected from the group consisting of alkylphosphates and arylphosphates.

5. The photo-hydrogel according to claim 4, wherein R¹ is selected from the group consisting of C₁-C₈ unbranched alkylphosphates, C₁-C₈ branched alkylphosphates, substituted monoarylphosphates, and unsubstituted monoarylphosphates.

6. The photo-hydrogel according to claim 1, wherein R² is selected from the group consisting of alkylsulfonates and arylsulfonates.

7. The photo-hydrogel according to claim 6, wherein R² is selected from the group consisting of C₁-C₈ unbranched alkylsulfonates, C₁-C₈ branched alkylsulfonates, substituted monoarylsulfonates, and unsubstituted monoarylsulfonates.

8. The photo-hydrogel according to claim 1, wherein R² is selected from the group consisting of alkylphosphates and arylphosphates.

9. The photo-hydrogel according to claim 8, wherein R² is selected from the group consisting of C₁-C₈ unbranched alkylphosphates, C₁-C₈ branched alkylphosphates, substituted monoarylphosphates, and unsubstituted monoarylphosphates.

10. The photo-hydrogel according to claim 1, wherein R² is selected from the group consisting of substituted polyarylsulfonates, unsubstituted polyarylsulfonates, substituted polyarylphosphates, and unsubstituted polyarylphosphates.

11. The photo-hydrogel according to claim 1, wherein R³ is selected from the group consisting of alkylammoniums and arylammoniums.

12. The photo-hydrogel according to claim 11, wherein R³ is selected from the group consisting of C₁-C₈ unbranched alkylammoniums, C₁-C₈ branched alkylammoniums, substituted monoarylammoniums, and unsubstituted monoarylammoniums.

13. The photo-hydrogel according to claim 1, wherein R⁴ is selected from the group consisting of alkylsulfonates and arylsulfonates.

14. The photo-hydrogel according to claim 13, wherein R⁴ is selected from the group consisting of C₁-C₈ unbranched alkylsulfonates, C₁-C₈ branched alkylsulfonates, substituted monoarylsulfonates, and unsubstituted monoarylsulfonates.

15. The photo-hydrogel according to claim 1, wherein R⁴ is selected from the group consisting of alkylphosphates and arylphosphates.

16. The photo-hydrogel according to claim 15, wherein R⁴ is selected from the group consisting of C₁-C₈ unbranched alkylphosphates, C₁-C₈ branched alkylphosphates, substituted monoarylphosphates, and unsubstituted monoarylphosphates.

17. The photo-hydrogel according to claim 1, wherein R⁴ is selected from the group consisting of substituted polyarylsulfonates, unsubstituted polyarylsulfonates, substituted polyarylphosphates, and unsubstituted polyarylphosphates.

18. The photo-hydrogel according to claim 1, wherein the polymer of formula (II) is configured as an actuator in at least one of a light-controlled micropump, artificial eye muscle, camera lens, tactile surface, or drug delivery system.

19. A photo-hydrogel comprising a polymer of formula (II): 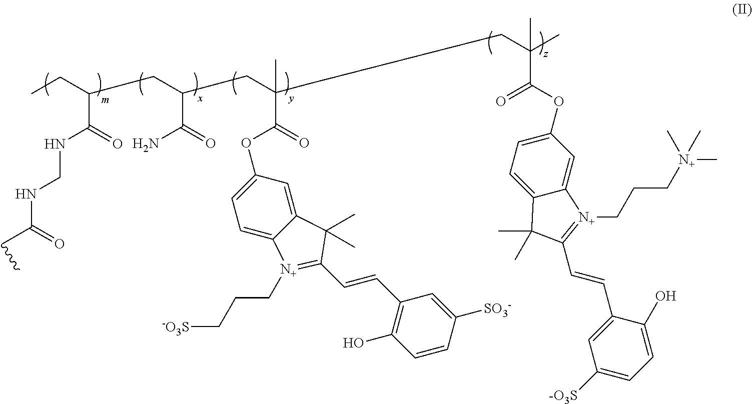
where when x=100, m<5 and x=y<16.

20. The photo-hydrogel according to claim 19, wherein the polymer of formula (II) is configured as an actuator in at least one of a light-controlled micropump, artificial eye muscle, camera lens, tactile surface, or drug delivery system.
